(19) United States
(12) Patent Application Publication
(10) Pub. No.: US 2022/0218869 A1
(43) Pub. Date: Jul. 14, 2022

(54) SPRAYABLE ANTIMICROBIAL COATINGS FOR WOUND TREATMENTS

(71) Applicant: 3M INNOVATIVE PROPERTIES COMPANY, St. Paul, MN (US)

(72) Inventors: Robert A. ASMUS, Hudson, WI (US); Patrick J. PARKS, Mendota Heights, MN (US); Shrijana B. SHRESTHA, Lakeville, MN (US)

(21) Appl. No.: 17/608,032

(22) PCT Filed: Apr. 28, 2020
(86) PCT No.: PCT/IB2020/054005

§ 371 Date: Nov. 1, 2021

Related U.S. Application Data
(60) Provisional application No. 62/842,242, filed on May 2, 2019.

(51) Int. Cl. A61L 26/00

(52) U.S. Cl. CPC A61L 26/0066; A61L 26/0076; A61L 2300/404; A61L 2300/206; A61L 2300/204; A61L 2300/802

(57) ABSTRACT

A composition for treating wounds is provided. The composition includes an antimicrobial component, an emulsifying agent, and a cidatrope component. The antimicrobial component can be selected from octenidine, poly(hexamethylene biguanide), a quaternary ammonium antimicrobial compound, and salts thereof. The cidatrope is not an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkalyl carboxylic acid, a phenolic compound, a (C1-C10)monohydric alkyl alcohol, or an ether glycol. In certain embodiments, the composition can be substantially free of water and C2-C5 lower alcohols. In certain embodiments, the composition further can comprise water. Methods of using the composition are also provided.

### TECHNICAL FIELD

The present disclosure relates to antimicrobial compositions containing an antimicrobial component, an emulsifier, and a cidatrope component , where the cidatrope component is not an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)monohydric alkyl alcohol, or an ether glycol.

### BACKGROUND

It is standard practice in the industrialized world to disinfect the skin prior to any invasive procedure such as, for example, surgery, catheterization, or needle puncture, to reduce the risk of infection. Various antimicrobial agents, e.g., cationic antimicrobial agents, are established in the marketplace for use as a disinfectant and antiseptic for skin disinfection before surgery and also for sterilizing surgical instruments and for cleaning wounds.

Some antimicrobial agents are used not only as an antiseptic to prevent hospital infections and as an adjuvant in oral hygiene, but also as a preservative in personal care products, such as, for example, antimicrobial dressings, skin preparations, bathing formulations, and nasal sprays.

### SUMMARY

The present disclosure provides compositions useful as products for skin disinfection such as skin antiseptics, preoperative surgical preps, hand sanitizers, catheter and i.v. skin preps, and waterless hand scrubs. The preferred formulations of the present invention, in general, facilitate reduction of microorganisms in a wound without significant cytotoxic effects on the healing tissue in the wound. Additionally, preferred formulations facilitate formation of a film that covers the wound to prevent further contamination by soil or microorganisms as the wound is healing. When used as an antiseptic in a nonclinical setting, the compositions described herein achieve improved antimicrobial efficacy with simultaneous tissue healing.

In one aspect, the present disclosure provides a composition. The composition can comprise an antimicrobial component, an emulsifying agent, and a cidatrope component. The antimicrobial component can be selected from the group consisting of octenidine, poly(hexamethylene biguanide), a quaternary ammonium antimicrobial compound, and a salt of any one of the foregoing antimicrobial components. The cidatrope is not an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)inonohydric alkyl alcohol, or an ether glycol. In any embodiment, the composition can be substantially free of water and C2-C5 lower alcohols. In any embodiment, the composition further can comprise water.

In any of the above embodiments, the composition further can comprise a polyhydric glycol having an average molecular mass of about 90-3000. In any of these embodiments, the polyhydric glycol can be selected from the group consisting of glycerol, diglycerol, polyglycerol-3, polyglycerol-4, polyglycerol-6, decaglycerol, polyglycerol-30, polyethylene glycol having a molecular weight of about 300-1000 daltons, propylene glycol, dipropylene glycol, an ethoxylate of sorbitol, an ethoxylate of glycerol and a combination of any two or more of the foregoing polyhydric glycols.

In any of the above embodiments, the cidatrope can comprise a molecule comprising at least 14 carbon atoms. In any of the above embodiments, the cidatrope component can be selected from the group consisting of polyoxyethylene (2) lauryl ether, octyl dodecyl neopentanate, acetyl triethyl citrate, propylene glycol monocaprylate, butyl tri-n-hexyl citrate, 2-octyldecanol, 2-butyloctanol, 2-butyldecanol, 2-hexyloctanol, isocetyl alcohol, isomyristyl alcohol, isoarachidyl alcohol, hexyldecanol, isostearyl alcohol, octyldodecanol, 2-butyloctanoic acid, hexyldecanoic acid, 1,2-octanediol, ethylhexylglycerin, diisopropyladipate, propylene glycol monolaurate, capryl pyrrollidone, lauryl pyrrolidone, and triethyl citrate.

In any of the above embodiments, the emulsifying agent can have a hydrophilic-lipophilic balance greater than 6. In any of the above embodiments, the emulsifying agent can be selected from the group consisting of decaglyceryl monolaurate, a polyoxyethylene sorbitan fatty acid ester, polyglyceryl-6 laurate, PEG-10 Phytosterol, PPG-4-Ceteth-20, hexaglycerol monolaurate, hexaglyceryl monomyristate, hexaglycerol monooleate, hexaglycerol monostearate, decaglycerol monolaurate, decaglyceryl monomyristate, decaglyceryl monooleate, decaglyceryl monostearate, a PEG sorbitol ester, a PEG sorbitan ester, a PEG alkyl ester, a PEG alkyl ether, PEG-5 Soya Sterol, PEG-10 Soya Sterol, PEG-20 Soya Sterol, PEG-30 Soya Sterol, PEG-25 Phytosterol, Dihdrocholeth-30, a PEG-PPG copolymers, an alkyl ether of PEG-PPG, and an alkyl polyglucoside.

In any of the above embodiments, the composition further can comprise a thickening agent. In any of the above embodiments, the composition further can comprise a mixture of metal salts comprising KCl, ZnCl₂, RbCl, and CaCl₂.

In another aspect, the present disclosure provides a method of treating a wound. The method can comprise applying the composition of any one of the preceding embodiments to a wound site. In any embodiment, applying the composition to the wound site can comprise spraying the composition onto the wound site. In any implementation, the method further can comprise applying a dressing to the wound site. In any of the above implementations of the method, the composition can be applied to the dressing before the dressing is applied to the wound site, wherein applying the dressing to the wound site comprises contacting the wound site with the composition applied to the dressing.

In yet another aspect, the present disclosure provides a kit. The kit can comprise a composition. The composition can comprise an antimicrobial component, an emulsifying agent, and a cidatrope component. The antimicrobial component can be selected from the group consisting of octenidine, poly(hexamethylene biguanide), a quaternary ammonium antimicrobial compound, and a salt of any one of the foregoing antimicrobial components. The cidatrope is not alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)monohydric alkyl alcohol, or an ether glycol. In any embodiment, the kit further can comprise a spray applicator or a dressing.

The terms “comprises” and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an affliction means preventing, treating, or both treating and preventing further afflictions).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION

“Ambient temperature” as used herein refers to the temperature range between about 21° and 25° C.

“Emollient” as used herein refers to materials which are capable of maintaining or improving the moisture level, compliance, or appearance of the skin when used repeatedly. Emollients often act to increase the moisture content of the stratum corneum. Emollients are generally separated into two broad classes based on their function. The first class of emollients function by forming an occlusive barrier, which reduces water evaporation from the stratum corneum. The first class of emollients is further subdivided into compounds, which are waxes at room temperature and compounds which are liquid or oils. The second class of emollients penetrate into the stratum corneum and physically bind water to prevent evaporation. The second class of emollients includes those that are water soluble and are often referred to as humectants. For the purposes of this invention, the emollient esters are considered separate and distinct from any other emollients which may be used, even though the emollient esters may function as occlusive emollients and aid in maintaining or improving the skin condition.

“Surfactant” as used herein is synonymous with “emulsifier,” and means an amphiphile (a molecule possessing both polar and nonpolar regions which are covalently bound) capable of reducing the surface tension of water and/or the interfacial tension between water and an immiscible liquid.

“Fatty” as used herein refers to a hydrocarbon chain length of 8 or more carbon atoms (odd or even number), unless otherwise specified

“Cidatrope” as used herein is a term for a hydrophobic component in the composition that enhances the effectiveness of the antimicrobial composition such that when the composition less the antimicrobial agent and the composition less the cidatrope component are used separately, they do not provide the same level of antimicrobial activity as the composition as a whole. For example, a cidatrope component in the absence of the antimicrobial agent may not provide any appreciable antimicrobial activity. The enhancing effect can be with respect to the level of kill, the speed of kill, and/or the spectrum of microorganisms killed, and may not be seen for all microorganisms. The cidatrope component may be a synergist such that when combined with the remainder of the composition, the composition as a whole displays an activity that is greater than the sum of the activity of the composition less the cidatrope component and the composition less the antimicrobial agent. The cidatrope preferably is a liquid at ambient conditions with a melt temperature less than 25° C. When more than one cidatrope is present in the antimicrobial composition, at least one cidatrope has a melt temperature less than 25° C. The hydrophobic emollient esters, and the optional fatty component all function as cidatropes in the compositions described herein.

“Hydrophobic” or “water insoluble” refers to a material that will not significantly dissolve in water at 23° C. Solubility can be determined by thoroughly mixing the compound with water at the appropriate concentration at 23° C. for at least 24 hours (or at elevated temperature if that is necessary to dissolve the compound), allowing this to sit at 23-25° C. for 24 hours, and observing the sample. In a glass jar with a 4-cm path length the sample should have evidence of a second phase, which can be liquid or solid and may be separated on the top, bottom, or distributed throughout the sample. For crystalline compounds care should be taken to avoid producing a supersaturated solution. The components should be mixed and observed. Cloudiness or presence of a visible precipitate or separate phase indicates that the solubility limit has been exceeded. Typically, when placed in 1×1 cm cell the sample has less than 70% transmission measured in a suitable spectrophotometer at a wavelength of 655 nm. For solubility determinations less than that which can be observed with the naked eye the solubility is determined using radiolabeled compounds as described under “Conventional Solubility 5 Estimations in Solubility of Long-Chain Fatty Acids in Phosphate Buffer at pH 7 .4,” Henrik Vorum, et al. in Biochimica et. Biophysica Acta, 1126, 135-142 (1992). The hydrophobic cidatropes of this invention have a solubility in water of less than 1%, more preferably less than 0.5%, even more preferably less than 0.25%, and most preferably less than 0.10%.

“Hydrophilic” or “water soluble” or “water swellable” refers to a material that will dissolve, solubilize, disperse or otherwise suspend in water (or other aqueous solution as specified) at a temperature of 23° C. in an amount of at least 7% by weight, preferably at least 10% by weight, more preferably at least 20% by weight, even more preferably at least 25% by weight, even more preferably at least 30% by weight, and most preferably at least 40% by weight, based on the total weight of the hydrophilic material and the water. The component is considered dissolved if after thoroughly mixing the compound with water at 60° C. for at least 4 hours and allowing this to cool to 23-25° C. for 24 hours, and mixing the composition thoroughly it appears uniform clear solution without visible cloudiness, phase separation, or precipitate in a jar having a path length of 4 cm. Typically, when placed in 1×1 cm cell, the sample exhibits greater than 70% transmission measured in a suitable spectrophotometer at a wavelength of 655 nm. Water dispersible hydrophilic materials disperse in water to form uniform cloudy dispersions after vigorous shaking of a 5% by weight mixture of the hydrophilic component in water. Water swellable hydrophilic materials solubilize or suspend in water, including those materials that form of a viscous solution or viscous gel.

“Cytotoxic effect”, as used herein, relates to an effect of one or more component of an antimicrobial composition that causes decreased viability of mammalian cells at a wound site.

“Nonvolatile” means that the component does not evaporate readily at ambient conditions, such that a 20-gm sample in a 4 cm² dish does not lose more than 2% of its weight, e.g., within 60 minutes upon exposure to ambient conditions. Examples of nonvolatile components of the compositions described herein include glycerin, chlorhexidine and its salts, and fatty components with a chain length greater than 10 carbons.

A composition of the present disclosure comprises an antimicrobial component. The antimicrobial component is effective to inhibit and/or kill microorganisms that can colonize or infect a wound site. Preferred antimicrobial components are effective to inhibit or kill bacterial that are known to form biofilms at a wound site. In addition, preferred antimicrobial components have relatively low cytotoxicity against mammalian cells when used in concentrations according to the present disclosure. Advantageously, the compositions of the present disclosure include components (e.g., a cidatrope) that enhances the antimicrobial activity of the antimicrobial component. In addition, the compositions include components (e.g., the cidatrope and/or the emulsifier) that reduce the inherent cytotoxic effect(s) of the antimicrobial component. Because of these advantages, it may be possible either maintain the antimicrobial effect while using a lower conc of antimicrobial in order to reduce cytotoxic effects in the wound or use a higher concentrations of antimicrobial components to boost the antimicrobial activity in the compositions without causing unacceptable cytotoxic effects on the healing tissue in a wound.

Antimicrobial agents useful in embodiments of the present disclosure may include cationic antimicrobial agents. The cationic antimicrobial agent is that component of the composition that provides at least part of the antimicrobial activity. That is, the cationic antimicrobial agent has at least some antimicrobial activity for at least one microorganism, e.g. Staphylococcus aureus. The cationic antimicrobial agent is generally considered the main active component of the compositions described herein. The cationic antimicrobial agent includes an effective amount of one or more antimicrobial agents selected from the group consisting of biguanides and bisbiguanides, such as chlorhexidine and its various salts including, but not limited to, the digluconate, diacetate, dimethosulfate, and dilactate salts, as well as combinations thereof; polymeric quaternary ammonium compounds such as Octenidine and its salts, polyhexamethylenebiguanide and its salts; small molecule quaternary ammonium compounds such as benzalkonium halides, benzethonium halides, alkyl substituted benzethonium halides, cetyl pyridinium halides; and compatible combinations thereof. It is particularly important, however, with cationic antimicrobial agents in a salt form to use a counter ion that ensures solubility in aqueous fluid above the minimum inhibitory concentration (“MIC”) of the treatment organism. If the solubility limit is less than the MIC, treatment may be ineffective.

The classes of cationic antimicrobial agent suitable in the present invention are discussed further below.

### Biguanides

This class of antimicrobials is represented by the formula:

R—NH—C(NH)—NH—C(NH)—NH(CH₂)ₙNHC(NH)—NH—C(NH)-—NH—R

where n=3-10, preferably 4-8, and most preferably 6; and R.=C₄-C₁₈ branched or straight chain alkyl optionally substituted in available positions by halogen or C₆-C₁₂ aryl or alkaryl optionally substituted in available positions by a halogen.

In some embodiments, the preferred compound of this class is chlorhexidine. This may be present as the free base or as a disalt of acetate, gluconate, lactate, methosulfate (CH₃OSO₃⁻), or a halide or combinations thereof. In some embodiments, the antimicrobial agent is chlorhexidine digluconate (“CHG”). Other anions may be useful.

Bis(biguanide)s such as chlorhexidine are very basic and capable of forming multiple ionic bonds with anionic materials. For this reason, biguanide-containing compositions are preferably free of anionic compounds that can result in precipitation of the antimicrobial. Anionic surfactants useful, for example, as wetting agents, may also need to be avoided. Halide salts may need to be avoided. For example, chlorhexidine digluconate (“CHG”) will precipitate rapidly in the presence of halide salts above a concentration of about 0.1 M.

### Polymeric Quaternary Amine Compounds

Antimicrobial polymers comprising quaternary amine groups may also be used as the cationic antimicrobial agent in the compositions described herein. These are typically polymers having quaternary amine groups with at least one alkyl or aralkyl chain of at least 6 carbon atoms and preferably as least 8 carbon atoms. The polymers may be linear, branched, hyperbranched or dendrimers. Preferred antimicrobial polymeric quaternary amine polymers include those described in U.S. Pat. Nos. 6,440,405; 5,408,022; and 5,084,096; PCT Publication No. WO/02102244; and Disinfection, Sterilization and Preservation, S. Block, 4^{th} ed., 1991, Chapter 13, Lea & Febiger.

A particularly preferred class of polymeric quaternary ammonium antimicrobial compounds are polybiguanides. Compounds of this class are represented by the formula:

X—R₁—NH—C(NH)—NH—C(NH)—NH—R²—NHC(NH)—NH—C(NH)—NH—R³—X

where R¹, R², and R³ are bridging groups such as polymethylene groups preferably having 2 to 10 methylene groups, more preferably 4 to 8 methylene groups and most preferably 6 methylene groups. The methylene groups can be optionally substituted in available positions with halogen, hydroxyl, or phenyl groups. X is a terminal group and is typically an amine, amine salt, or a dicyandiamide group. The preferred compound of this class is polyhexamethylene biguanide (“PHMB”) commercially available as COSMOCIL® CQ from Aveci, Wilmington, Del., USA.

Poly(biguanide) antimicrobials such as PHMB are very basic and are capable of forming multiple ionic bonds with anionic materials. For this reason, biguanide-containing compositions are preferably free of anionic compounds that can result in precipitation and/or inactivation of the antimicrobial. Anionic surfactants useful, for example, as wetting agents, may also need to be avoided. Halide salts also may need to be avoided.

### Small Molecule Quaternary Ammonium Compounds

This class of compounds typically comprise one or more quaternary ammonium groups wherein attached to the quaternary ammonium group is at least one C₆-C₁₈ linear or branched alkyl or aralkyl chain. Suitable compounds include those disclosed in Disinfection, Sterilization and Preservation, S. Block, 4^{th} ed., 1991, Chapter 13, Lea & Febiger. Some compounds of this class have one or two C₈-C₁₈ alkyl or aralkyl chains and may be represented by the following formula:

R¹R²NR³R⁴⁺X⁻

where R¹ and R² are C₁-C₁₈ linear or branched alkyl, alkaryl, or aralkyl chains that may be substituted in available positions by N, O, or S provided at least one R¹ or R² is a C₈-C₁₈ linear or branched alkyl, alkaryl, or aralkyl chains that may be substituted in available positions by N, O, or S. R³ and R⁴ are C₁-C₆ alkyl, phenyl, benzyl, or C₈-C₁₂ alkaryl groups. R³ and R⁴ may also form a ring such as a pyridine ring with the nitrogen of the quaternary ammonium group. X is an anion, preferably a halide, and most preferably C₁— or Br—. Other anions may include methosulfate, ethosulfate, phosphates, and the like. Preferred compounds of this class include monalkyltrimethylammonium salts, monalkyldimethylbenzyl ammonium salts, dialkyldimethyl ammonium salts, and benzethonium chloride.

For certain embodiments of the antimicrobial composition, the antimicrobial component is selected from the group consisting of chlorhexidine, chlorhexidine digluconate, chlorhexidine diacetate, chlorhexidine dimethosulfate, chlorhexidine dilactate salts, polyhexamethylenebiguanide, benzalkonium halides, octenidine salts, and combinations thereof

For certain preferred embodiments of the antimicrobial composition, the antimicrobial component is selected from the group consisting of octenidine dihydrochloride, octenidine gluconate, octenidine sulfate, octenidine acetate, Octenidine methyl sulfate and combinations thereof.

For certain embodiments of the composition of the present disclosure, the antimicrobial component is selected from the group consisting of octenidine, poly(hexamethylene biguanide), a quaternary ammonium antimicrobial compound, and a salt of any one of the foregoing antimicrobial components. Nonlimiting examples of suitable quaternary ammonium compounds include Alexidine, Benzalkonium Chloride, Benzethonium Chloride, Benzyldimethylhexadecylammonium Chloride, Benzyldimethyltetradecylammonium Chloride, Cetylpyridinium Chloride, Cetyltrimethylammonium Bromide, Cetyltrimethylammonium p-Toluenesulfonate, Chloramine-T Trihydrate, Dequalinium Chloride, Dodecyltrimethylammonium Bromide, Dodecyltrimethylammonium Chloride, Domiphen Bromide, Ethylhexadecyldimethylammonium Bromide, Hexadecyltrimethylammonium Chloride, Hexamidine Diisethionate, Icthammol, Methylbenzethonium Chloride, and Tetradecyltrimethylammonium Bromide.

The antimicrobial component is at least 0.1 wt. %, at least 0.5 wt. %, at least 1 wt. %, or at least 1.5 wt. % based on the total weight of the dried composition. The antimicrobial component is commonly no more than 10 wt. %, no more than 8 wt. %, no more than 6 wt. %, no more than 4 wt. %, based on the total weight of components in the composition. The antimicrobial component is commonly 0.1 wt. % to 10 wt. %, 0.5 wt. % to 8 wt. %, 1 wt. % to 6 wt. %, or 1.5 wt. % to 4 wt. %, based on the total weight of components in the composition.

A composition of the present disclosure further comprises an emulsifying agent. Without being bound by theory, the inventors believe the emulsifying agent in combination with the cidatrope, facilitates formation of micelles, which enhance the delivery of the antimicrobial component (e.g., PHMB) to the wound site while minimizing the toxicity (to the wound tissue) of the antimicrobial component. Accordingly, the compositions of the present disclosure afford greater microbial inhibition and/or microbicidal activity at relatively lower concentrations of antimicrobial component.

In certain embodiments, the emulsifier used in the composition has a hydrophilic-lipophilic balance (HLB) greater than 6. In certain embodiments, the emulsifier used in the composition has a hydrophilic-lipophilic balance (HLB) greater than 8. In certain embodiments, the emulsifier used in the composition has a hydrophilic-lipophilic balance (HLB) greater than 10. Nonlimiting examples of suitable emulsifiers include decaglyceryl monolaurate, a polyoxyethylene sorbitan fatty acid ester, polyglyceryl-6 laurate, PEG-10 Phytosterol, PPG-4-Ceteth-20, hexaglycerol monolaurate, hexaglyceryl monomyristate, hexaglycerol monooleate, hexaglycerol monostearate, decaglycerol monolaurate, decaglyceryl monomyristate, decaglyceryl monooleate, decaglyceryl monostearate, a PEG sorbitol ester, a PEG sorbitan ester, a PEG alkyl ester, a PEG alkyl ether, PEG-5 Soya Sterol, PEG-10 Soya Sterol, PEG-20 Soya Sterol, PEG-30 Soya Sterol, PEG-25 Phytosterol, Dihdrocholeth-30, a PEG-PPG copolymers, an alkyl ether of PEG-PPG, and an alkyl polvglucoside.

A composition of the present disclosure further comprises cidatrope component. Cidatropes suitable for use in compositions of the present disclosure include C₈-C₂₆ alcohols, ethers, amides, esters, and combinations thereof. In some embodiments, the C₈-C₂₆ alcohol cidatrope is selected from the group consisting of 1-tetradecanol, hexadecanol, 16-methyl-1-heptadecanol, and combinations thereof. In some embodiments, the ether cidatrope is a propoxylated C₂ to C₁₈ alcohol having a degree of propoxylation of 2 to 50 moles per mole of alcohol. In some embodiments, the amide cidatrope is selected from the group consisting of a coconut fatty acid monoethanol amide, a coconut fatty acid methyl ethanolamide, an alkyl alkanolamide, and combinations thereof In some embodiments, the ester cidatrope is selected from the group consisting of diisopropyl adipate, dibutyl sebacate, triethyl citrate, tributyl citrate, octyldodecyl neopentanoate, laureth-2-acetate, isopropyl myristate, trioctyldodecyl citrate, myristyl myristate, cetyl acetate, and combinations thereof.

Cidatrope components of the present disclosure do not include an alpha-hydroxy acid, a beta-hydroxy acid, a chelating agent, a (C1-C₄)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)monohydric alkyl alcohol, or an ether glycol.

The cidatrope component is at least 0.5 wt. %, at least 1 wt. %, at least 2.5 wt. %, or at least 5 wt. % based on the total weight of the dried composition. The cidatrope is commonly no more than 99.4 wt. %, no more than 95 wt. %, no more than 90 wt. %, or no more than 85 wt. %, based on the total weight of nonvolatile components in the composition. The solubilizer is commonly 60 wt. % to 99.4 wt. %, 65 wt. % to 95 wt. %, 70 wt. % to 90 wt. %, or 75 wt. % to 85 wt. %, based on the total weight of nonvolatile components in the composition.

Antimicrobial compositions of the present disclosure may be prepared by methods known in the art. For example, the antimicrobial agent, solubilizer, and cidatrope may be combined, either stepwise or all at once, in a suitable container to provide a mixture. In some embodiments, the antimicrobial agent, solubilizer, and cidatrope may be combined at room temperature (e.g., 23°). In some embodiments, one or more of the antimicrobial agent, solubilizer, and cidatrope may be heated and/or melted before combination with other components of the antimicrobial composition. The mixture may be stirred or otherwise agitated for a period of time (e.g., 24 hours) to provide a homogenous antimicrobial composition.

Antimicrobial compositions of the present disclosure may be useful to prevent hospital infections as a preoperative surgical, catheter, or intravenous, as an adjuvant in oral hygiene, and in personal care products, such as, for example, antimicrobial dressings, skin preparations, bathing formulations, and nasal sprays.

In certain embodiments of an antimicrobial composition of the present disclosure, the composition is substantially free of water. In certain embodiments of an antimicrobial composition of the present disclosure, the composition is substantially free of C2-C5 lower alcohols.

In certain embodiments, an antimicrobial composition of the present disclosure optionally comprises a polyhydric glycol having an average molecular mass of about 90-3000. The polyhydric alcohol helps maintain hydration of the skin and formulation which facilitates antimicrobial activity. Suitable polyhydric alcohols include, but are not limited to glycerol, diglycerol, polyglycerol-3, polyglycerol-4, polyglycerol-6, decaglycerol, polyglycerol-30, polyethylene glycol having a molecular weight of about 300-1000 daltons, propylene glycol, dipropylene glycol, an ethoxylate of sorbitol, an ethoxylate of glycerol and a combination of any two or more of the foregoing polyhydric glycols.

In certain embodiments, an antimicrobial composition of the present disclosure optionally comprises a thickening agent. The thickening agent can be hydrophilic polymer or an associative thickener. Nonlimiting examples of suitable thickening agents include a hydrophilic polymer selected from the group consisting of cross-linked polyvinylpyrrolidone (PVP), polyvinyl alcohol (PVA), Guar gum, cellulose, and water-soluble or water-dispersible derivatives thereof.

In certain embodiments, an antimicrobial composition according to the present disclosure further comprises a mixture of metal salts comprising KC1, ZnCl₂, RbCl, and CaCl₂. The mixture of metal salts facilitates the release of biologic biomarker responses which facilitate healing of the wound tissue.

In any embodiment, an aqueous antimicrobial composition of the present disclosure has a pH of about 4 to about 9; more preferably, about 5 to about 8; and even more preferably, about 6 to about 7. Anhydrous antimicrobial compositions of the present disclosure, when mixed 1:1 with deionized water have a pH of about 4 to about 9; more preferably, about 5 to about 8; and even more preferably, about 6 to about 7. Optionally, a composition of the present disclosure may include a buffering agent.

### Methods of Preparation

The compositions of the present invention may be prepared by a variety of techniques. The processing variables including amount and intensity of high shear mixing, rate of cooling, and order of addition are easily determined by one skilled in the art.

In another aspect, the present disclosure provides a method of treating a wound. The method comprises applying to a wound site the composition of any one of the embodiments of the antimicrobial composition of the present disclosure. In certain implementations, applying the antimicrobial composition comprises spraying the composition onto the wound site.

In certain implementations, a method of treating a wound according to the present disclosure further comprises applying a wound dressing to the wound site. In some implementations, the antimicrobial composition is applied to the wound dressing before the dressing is applied to the wound site, wherein applying the dressing to the wound site comprises contacting the wound site with the composition applied to the dressing.

In yet another aspect, the present disclosure provides a kit. The kit includes the composition of any one of the embodiments of the antimicrobial composition according to the present disclosure. In any embodiment, the kit further comprises a spray applicator for applying the antimicrobial composition to a wound or to a wound dressing. In any embodiment, the kit further comprises a wound dressing.

### TEST METHODS

Compositions of the present disclosure can be evaluated for efficacy by testing several parameters—antimicrobial activity and tissue cytotoxicity. Preferred compositions have relatively high antimicrobial activity (bactericidal and/or bacteriostatic) while causing relatively low cytotoxicity to healing tissue at a mammalian wound site.

### Test Method 1. Ex Vivo Human Skin Biofilm Assay

Tissue preparation: Human tissue used in the experiments is stored in HYPOTHERMOSOL® preservation medium. Excess adipose tissue is trimmed from the bottom of the tissue. The skin is placed in a paraffin tray and the corners of the tissue are pinned to the paraffin using sterile pins. Excess HYPOTHERMOSOL is blotted from the tissue surface with paper towels. Wounds (2 mm diameter) are made on the surface of the tissue using tweezers and a scalpel to cut away the epidermis. A 5 mm diameter punch (biopsy) is made around the 2 mm wound, going deeper into the dermis. Tweezers and a scalpel are used to remove the wounded biopsy from the skin. The biopsy with the wound is placed in RPMI medium containing 2% human sera. A 6-well plate with is prepared by placing 2.0±0.5 mL RPMI medium containing 10% FBS and a transwell insert into each well. Tissue biopsy explants (three per well) are placed wound-side up into the inserts.

Bacteria prep: The bacterial strains used in these experiments are Acinetobacter baumannii (QE613), Pseudomonas aeruginosa (C3), Methicillin-Resistant Staphylococcus aureus (USA300LAC), Methicillin-Resistant Staphylococcus aureus (C3) obtained from clinical isolates.

A culture tube containing Todd-Hewitt (TH) broth is inoculated with multiple colonies (from TSA agar) of the test microorganism. The inoculated broth tube is placed into shaking incubator, (37±2° C., 200±50 rpm) and incubated for approximately 18 hours. After incubation, a 1±0.1 mL portion of the broth culture is placed in a sterile microcentrifuge tube. The portion is centrifuged (1±0.5 min at max speed) to pellet the bacteria. The pellet is washed with 1±0.1 mL RPMI medium. The cells are pelleted by centrifugation and resuspended in 1±0.1 mL fresh RPMI medium. A portion of the resuspended cells (300±20 □) is diluted into 5±0.5 mL fresh RPMI. A portion of the diluted, resuspended cells (2±1□1 per explant) is added to each well and the microwell plates are returned to incubator for 1-5 days.

Treatment: Antimicrobial compositions (100±15□1) are added to each microwell using a syringe. After adding the composition, the microwell plates are incubated at 37±2° C. for 24±4 h.

Sampling: After incubation with the antimicrobial composition, the biopsy explants are transferred into 250±20 □L Standard Sample Solution (“SSS”; 6.0 g lecithin, 50.0 g Tween 80, 0.4 g KH₂PO₄, 10.1g Na₂HPO₄, 1.0 g Triton X-100, 1.0 g sodium thiosulfate, and 4.0 g Poly(sodium 4-styrenesulfonate) plus water to a final volume of 1000 mL). The samples are vortex-mixed for 30±10 seconds and then sonicated for 2±0.5 minutes. The sonicates are vortex-mixed for 30±10 seconds. The resulting mixtures are diluted in phosphate-buffered saline (PBS) and aliquots of the dilutions are spread onto TSA+5% SB and incubated at 37±2° C. The remainder of the dilutions are stored at 4±2° C. Colonies on each of the plates are counted the following day. If the colonies on the highest dilutions are too numerous to count, the remainder of the stored suspensions are diluted further, spread on TSA +5% SB, incubated and the colonies are counted as described above.

### Test Method 2: Ex Vivo Porcine Mucosal Tissue Biofilm Assay

Tissue Prep: The tissue is trimmed as described in Test Method 1 and then collected in RPMI 1640 medium+5% penicillin/streptomycin solution (part# P4458 obtained from Sigma-Aldrich, St. Louis, Mo.). Biopsy punches (5 mm diameter) are prepared as described in Test Method 1 to produce the explants for this assay. Most of the remaining muscle tissue is removed with a fresh scalpel blade. The explants are rinsed three times with 10±2 ml RPMI (no antibiotics, no Fetal Calf Serum). Explants are covered with fresh media placed in incubator for ˜30 min. A 6-well plate is prepared with 2.0±0.5 mL RPMI (no antibiotics, no Fetal Calf Serum) in the wells and a transwell insert is placed in each well. Tissue explants are transferred mucosal side-up to the transwell inserts (3 explants/well).

Bacteria Prep: A fresh agar culture of each bacterial strain is from frozen stock is prepared within two weeks of the experiment. A culture tube containing Todd Hewitt broth is inoculated with several colonies and placed into shaking incubator, (37±2° C., 200±50 rpm) overnight. A 1±0.1 mL portion of the overnight culture is removed from the overnight culture and placed into a sterile microcentrifuge tube. The microcentrifuge tube is centrifuged (1±0.5 min at max speed) to pellet the bacteria. The pellet is washed with 1±0.1 mL RPMI, no ABX, no FCS.

After washing, the pellet is resuspended in 1±0.1 mL of fresh RPMI. A 300±20□1 portion of the resuspended cells is diluted into 5±0.5 mL of fresh RPMI. The resulting diluted bacterial suspensions (2±1□1 per explant) are added to each transwell insert and the 6-well plates are returned to the 37° C. incubator for 1-5 days.

Treatment: Antimicrobial compositions (100±15□1) are added to each microwell using a syringe. After adding the composition, the microwell plates are incubated at 37±2° C. for 24±4 h.

Sampling: The explants are transferred into 250±20□L Standard Sampling Solution and the vortex mixed for 30±10 seconds, sonicated for 2±0.5 minutes, and then vortex mixed for another 30±10 seconds. The resulting sonicates are plated on TSA +5% Sheep Blood to determine colony counts as described in Test Method 1.

### Test Method 3. Ex Vivo Porcine Mucosal Tissue Cytotoxicity Assay

Tissue Prep: The tissue is trimmed as described in Test Method 1 and then collected in RPMI 1640 medium. A 5 mm biopsy punch is used to produce the explants for this assay. Most of the remaining muscle tissue is removed with a fresh scalpel blade. The explants are rinsed three times with 10±2 ml RPMI no ABX, no FCS. The wells of a 6-well plate are loaded with 2.0±0.5 mL RPMI (no ABX, no FCS) and a transwell insert is placed into each well. Tissue explants are transferred mucosal side-up into each of the inserts (3 explants/well).

Treatment: Antimicrobial compositions (100±15□1) are added to each microwell using a syringe. After adding the composition, the microwell plates are incubated at 37±2° C. for 24±4 h.

Assay for viability: The explants are rinsed three times with 1.0±0.2 mL RPMI. Each well of a 96-well plate is loaded with 100±15□L RPMI+10±2□L MTT substrate/well. The explants are transferred to the 96-well plate (1 explant/well). The 96-well plate is incubated at 37±2° C. for 1-3 h until purple color is well-developed on untreated explant controls. Each explant is transferred to 100±15□L of extraction reagent (isopropanol, acidified) is added to each well and the plate and is stored overnight at 4±2° C.

Measure O.D.: The explants are removed from 96-well plate and discarded. The optical density of each microwell is measured at 570 nm and 690 nm using a plate reader. The data are compared to the untreated controls, after subtracting out background controls, to calculate percent viability.

### Test Method 4. Ex Vivo Human Skin Cytotoxicity Assay

Human tissue used in the experiments is stored in HYPOTHERMOSOL preservation medium. Excess adipose tissue is trimmed from the bottom of the tissue. The skin is placed in a paraffin tray and the corners of the tissue are pinned to the paraffin using sterile pins. Excess HYPOTHERMOSOL medium is blotted from the tissue surface with paper towels. Wounds (2 mm diameter) are made on the surface of the tissue using tweezers and a scalpel to cut away the epidermis. A 5 mm diameter punch (biopsy) is made around the 2 mm wound, going deeper into the dermis. Tweezers and a scalpel are used to remove the wounded biopsy from the skin. The biopsy with the wound is placed in RPMI medium containing 2% human sera. A 6-well plate with is prepared by placing 2.0±0.5 mL RPMI medium containing 10% FBS and a transwell insert into each well. Tissue biopsy explants (three per well) are placed wound-side up into the inserts.

Treatment: Antimicrobial compositions (100±15□1) are added to each microwell using a syringe. After adding the composition, the microwell plates are incubated at 37±2° C. for 24±4 h.

Assay for viability: Rinse explants 3× with 1.0±0.2 mL RPMI. Set up 96-well non-tissue culture tray with 100±15 □L RPMI+10±2 □L MTT substrate/well. Transfer explants to 96-well plate: 1 explant/well. Return to 37±2° C. and incubate for 1-3 h until purple color is well developed on untreated explants. Transfer to wells containing 100±15 □L of extraction reagent (isopropanol, acidified). Store overnight at 4±2° C.

Assay for viability: The explants are rinsed three times with 1.0±0.2 mL RPMI. Each well of a 96-well plate is loaded with 100±15 □L RPMI +10±2 □L MTT substrate/well. The explants are transferred to the 96-well plate (1 explant/well). The 96-well plate is incubated at 37±2 ° C. for 1-3 h until purple color is well-developed on untreated explant controls. Each explant is transferred to 100±15 □L of extraction reagent (isopropanol, acidified) 100±15 □L of extraction reagent (isopropanol, acidified) is added to each well and the plate and is stored overnight at 4±2° C.

Measure O.D.: The explants are removed from 96-well plate and discarded. The optical density of each microwell is measured at 570 nm and 690 nm using a plate reader. The data are compared to the untreated controls, after subtracting out background controls, to calculate percent viability.

Advantages of this disclosure are further illustrated by the following non-limiting examples, but the particular materials and amounts thereof recited in these examples, as well as other conditions and details, should not be construed to unduly limit this disclosure.

### EXAMPLES

TABLE 1
Materials used in the Examples

| Name | Chemical Name | Source |
|---|---|---|
| AM-3130N | Cocamidopropyl Betaine | Nikko Chemical Co.; Tokyo, JP |
| Barcleanse LEP | Laureth-1 Phosphate | Barnet Products; Englewood Cliffs, NJ |
| BPS-10 | PEG-10 Soya Sterol | Nikko Chemical Co. |
| CDS-6000P | PEG-150 Distearate | Nikko Chemical Co. |
| COSMOCIL CQ RL_20 | 20% PHMB in water | Lonza Group; Portsmouth, NH |
| Decaglyn 1L | Decaglycerol monolaurate | Nikko Chemical Co. |
| Dextrin Palmitate | Dextrin Palmitate | Nikko Chemical Co. |
| DOP-8N | Dioleth-8 Phosphate | Nikko Chemical Co. |
| ECT-3NEX | Sodium Trideceth-3 Carboxylate | Nikko Chemical Co. |
| GLUCOPON ® 600UP | Lauryl Glucoside | BASF Corp.; St. Louis, MO |
| Hexaglyn-1L | Hexaglycerol Monolaurate | Nikko Chemical Co. |
| ISOFOL 18T | Isostearyl Alcohol | Sasol Chemicals (USA) LLC; Houston, TX |
| JARCOL ™ I-12 | Butyloctanol | Jarchem Innovative Ingredients |
| JARCOL I-14 | 2-Hexyl Octanol | Jarchem Innovative Ingredients |
| JARCOL I-15 | 2-Hexyl Decanol | Jarchem Innovative Ingredients |
| JARCOL I-18CG | Isostearyl alcohol | Jarchem Innovative Ingredients |
| JARCOL I-20 | 2-Octyl decanol | Jarchem Innovative Ingredients |
| JARCOL 8 | Capryl Alcohol | Jarchem Innovative Ingredients |
| JARIC ™ I-16 | 2-Hexyldecanoic acid | Jarchem Innovative Ingredients |
| Lecinol S-10 | Lecinol Phosphate | Nikko Chemical Co. |
| LMT | Sodium Methyl Lauroyl Taurate | Nikko Chemical Co. |
| LSA-F | Sodium Lauryl Sulfoacetate | Nikko Chemical Co. |
| PBC-33 | PPG-4-Ceteth-10 | Nikko Chemical Co. |
| PBC-34 | PPG-4-Ceteth-20 | Nikko Chemical Co. |
| SC50 | ethylhexylglycerin | Schulke; Fairfield, NJ |
| TL-10 | PEG-20 Sorbitan monolaurate | Nikko Chemical Co. |
| Tween 65 | POE-20 Sorbitan Tristearate | Croda Inc.; Edison, NJ |

All chemicals used in the Examples were reagent-grade, if available, unless otherwise noted.

### Examples 1-28. Antimicrobial Compositions Comprising PHMB with Various Cidatropes

Compositions were made by adding the components to a suitably-sized glass jar. polyhydric alcohol (e.g., polyglycerol), if used in the composition, was added to the jar first: followed by the emulsifier, the cidatrope, the antimicrobial component (e.g., PHMB) and then half of the water. Just before mixing this solution, the thickener (e.g., Hydroxy propyl guar), if used. was added and the composition was immediately mixed using a SILVERSON® homogenizer. As the mixture got more viscous, the remaining water was added slowly. Mixing was complete when there were no observable lumps and the aqueous mixture appeared homogeneous. The metal salts mixture, if used, was added with the water. The compositions of these Examples are listed in Table 2.

### Comparative Examples 1-3

The compositions for Comparative Examples 1 and 2 are shown in Table 2. They were prepared as described for Examples 1-28 except that neither of them comprised a cidatrope. In addition, Comparative Example 2 did not include an antimicrobial component.

TABLE 2
Antimicrobial compositions with various cidatropes. All of the compositions in Table
1 were prepared using the same antimicrobial component (polyhexamethylene biguanide
(PHMB)), the same emulsifier (decaglyceryl monolaurate), the same optional thickener
(cross-linked polyvinylpyrrolidone), and the same optional polyhydric alcohol (polyglycerol-
3). All compositions were prepared in 60-gram quantities.

| | | | | Decaglyceryl | Cross-linked | |
|---|---|---|---|---|---|---|
| | PHMB | | Cidatrope | Monolaurate | PVP | Polyglycerol-3 |
| Example | (wt %) | Cidatrope | (wt %) | (wt %) | (wt %) | (wt %) |
| Example 1 | 0.40 | Octyl dodecyl neopentanate | 0.50 | 0.75 | 3.00 | 15 |
| Example 2 | 0.40 | SC50 | 0.50 | 0.75 | 3.00 | 15 |
| Example 3 | 0.40 | Acetyl triethyl citrate | 0.50 | 0.75 | 3.00 | 15 |
| Example 4 | 0.40 | BL-2 (POE-2 Lauryl Ether) | 0.50 | 0.75 | 3.00 | 15 |
| Example 5 | 0.40 | CAPMUL ® 708G (glycerol | 0.50 | 0.75 | 3.00 | 15 |
| | | monocaprylate) | | | | |
| Example 6 | 0.40 | CAPMUL PG-8 (Propylene | 0.50 | 0.75 | 3.00 | 15 |
| | | Glycol Monocaprylate) | | | | |
| Example 7 | 0.40 | CITROFLEX ® B-6 (Butyl | 0.50 | 0.75 | 3.00 | 15 |
| | | tri-n-hexyl citrate) | | | | |
| Example 8 | 0.40 | ISOFOL 18T | 0.50 | 0.75 | 3.00 | 15 |
| Example 9 | 0.40 | JARCOL I-12 | 0.50 | 0.75 | 3.00 | 15 |
| Example 10 | 0.40 | JARCOL I-14 | 0.50 | 0.75 | 3.00 | 15 |
| Example 11 | 0.40 | JARCOL I-16 | 0.50 | 0.75 | 3.00 | 15 |
| Example 12 | 0.40 | JARCOL I-18CG | 0.50 | 0.75 | 3.00 | 15 |
| Example 13 | 0.40 | JARCOL I-20 | 0.50 | 0.75 | 3.00 | 15 |
| Example 14 | 0.40 | JARCOL 8 | 0.50 | 0.75 | 3.00 | 15 |
| Example 15 | 0.40 | JARIC I-12 | 0.50 | 0.75 | 3.00 | 15 |
| Example 16 | 0.40 | JARIC I-16 | 0.50 | 0.75 | 3.00 | 15 |
| Example 17 | 0.40 | Lexgard O (1,2-Octanediol) | 0.50 | 0.75 | 3.00 | 15 |
| Example 18 | 0.40 | Lexgard OE70 (1,2-Octanediol: | 0.50 | 0.75 | 3.00 | 15 |
| | | Ethylhexylglycerin 70:30) | | | | |
| Example 19 | 0.40 | PBC-31 (PPG-4 Ceteth-1) | 0.50 | 0.75 | 3.00 | 15 |
| Example 20 | 0.40 | PBC-41 (PPG-8 Ceteth-1) | 0.50 | 0.75 | 3.00 | 15 |
| Example 21 | 0.40 | Pelemol DIA (Diisopropyl | 0.50 | 0.75 | 3.00 | 15 |
| | | Adipate) | | | | |
| Example 22 | 0.40 | Propylene Glycol monolaurate | 0.50 | 0.75 | 3.00 | 15 |
| Example 23 | 0.40 | Surfadone ™ LP-100 (capryl | 0.50 | 0.75 | 3.00 | 15 |
| | | Pyrrollidone) | | | | |
| Example 24 | 0.40 | Surfadone ™ LP-300 (Lauryl | 0.50 | 0.75 | 3.00 | 15 |
| | | Pyrrollidone) | | | | |
| Example 25 | 0.40 | Triethyl citrate | 0.50 | 0.75 | 3.00 | 15 |
| Example 26 | 0.40 | Octyl dodecyl neopentanate plus | 0.50 | 0.75 | 3.00 | 15 |
| | | 0.05% Monosodium Phosphate | | | | |
| Example 27 | 0.40 | Octyl dodecyl neopentanate plus | 0.50 | 0.75 | 3.00 | 15 |
| | | 0.10% Monosodium Phosphate | | | | |
| Example 28 | 0.40 | Octyl dodecyl neopentanate plus | 0.50 | 0.75 | 3.00 | 15 |
| | | 0.20% Monosodium Phosphate | | | | |
| Comparative | 0.40 | None | 0.50 | 0.75 | 3.00 | 15 |
| Example 1 | | | | | | |
| Comparative | 0.00 | None | 0.50 | 0.75 | 3.00 | 15 |
| Example 2 | | | | | | |

### Antimicrobial and Cytotoxic Effects of the Compositions of Examples 1-28

The compositions of Examples 1-28 were tested according to Test Method 2 and Test Method 3 described hereinabove. The bacterial strain used in Test Method 2 was Methicillin-Resistant Staphylococcus aureus (C3). The biofilms were incubated for 72 hours before contact with the test compositions. The antimicrobial and cytotoxic activities of the compositions of Examples 1-28 were compared to a commercial antimicrobial wound gel (PRONTOSAN® Wound Gel) available from B. Braun (Bethlehem, Pa.). According to the product literature, PRONTOSAN Wound Gel contains Glycerol, Hydroxyethylcellulose, Undecylenamidopropyl Betaine (surfactant), and Polyaminopropyl Biguanide (disinfectant). The results of the tests are shown in Table 3.

TABLE 3
Antimicrobial and Cytotoxic Effects
of the Compositions of Examples 1-28.

| | | PVM |
|---|---|---|
| Example | LRV¹ | Cytotoxicity² |
| Example 1 | 5.09 | 93.10 |
| Example 2 | ND | ND |
| Example 3 | 5.32 | 84.01 |
| Example 4 | ND | ND |
| Example 5 | ND | ND |
| Example 6 | ND | ND |
| Example 7 | 4.99 | 102.09 |
| Example 8 | 4.54 | 94.79 |
| Example 9 | ND | ND |
| Example 10 | ND | ND |
| Example 11 | 5.91 | 85.64 |
| Example 12 | 5.45 | 86.66 |
| Example 13 | 5.83 | 83.05 |
| Example 14 | ND | ND |
| Example 15 | ND | ND |
| Example 16 | ND | ND |
| Example 17 | ND | ND |
| Example 18 | ND | ND |
| Example 19 | 4.84 | 73.34 |
| Example 20 | ND | ND |
| Example 21 | 4.31 | 83.64 |
| Example 22 | ND | ND |
| Example 23 | ND | ND |
| Example 24 | 5.20 | 69.09 |
| Example 25 | 5.46 | 83.77 |
| Example 26 | 4.74 | 83.32 |
| Example 27 | 5.75 | 82.83 |
| Example 28 | 5.77 | 80.13 |
| Comparative Example 1 | 5.63 | 62.91 |
| Comparative Example 2 | 0.55 | 110.45 |
| PRONTOSAN Wound Gel | 2.94 | 55.91 |

¹LRV = Log Reduction Value. Determined according to Test Method 2. Colony counts from biofilms treated with antimicrobial compositions were compared to colony counts from untreated biofilms (control). The difference between control and the test compositions was reported as the LRV.
²Cytotoxicity results are reported as percent viability of the cells in the explants of Test Method 3.

The results indicate the composition with no antimicrobial component or cidatrope (Comparative Example 2) had very little antimicrobial or cytotoxic effects. In addition, the composition with the antimicrobial component but no cidatrope (Comparative Example 2) had an antimicrobial effect but was moderately cytotoxic. The data also show a number of cidatrope-containing compositions that exhibited good antimicrobial activity and markedly less cytotoxic affects.

### Examples 29-47. Antimicrobial Compositions Comprising Octenidine with Various Cidatropes

The compositions of Examples 29-47 were prepared as described above for Examples 1-28 with the exception that the antimicrobial component used in these Examples was Octenidine instead of PHMB. The compositions of these Examples are listed in Table 4.

TABLE 4
Antimicrobial compositions with various cidatropes. All of the compositions in Table 1 were prepared using the same antimicrobial
component (Octenidine), the same emulsifier (decaglyceryl monolaurate), the same optional thickener (Hydroxypropyl Guar),
and the same optional polyhydric alcohol (polyglycerol-3). All compositions were prepared in 60-gram quantities.

| | Hydroxypropyl | | | Cidatrope | Octenidine | Decaglyceryl | |
|---|---|---|---|---|---|---|---|
| Example | Guar | Polyglycerol-3 | Cidatrope | (g) | (1% soln.) | Monolaurate | Water |
| Example 29 | 0.84 | 9.0 | SC50 | 0.3 | 6.0 | 0.45 | 43.41 |
| Example 30 | 0.84 | 9.0 | PPG-3 Myristyl ether, promyristyl PM-3 | 0.30 | 6.0 | 0.45 | 43.41 |
| Example 31 | 0.84 | 9.0 | PPG-10 Cetyl ether Procetyl 10 | 0.30 | 6.0 | 0.45 | 43.41 |
| Example 32 | 0.84 | 9.0 | Propylene Glycol Diperlargonate | 0.30 | 6.0 | 0.45 | 43.41 |
| Example 33 | 0.84 | 9.0 | Laureth-2 Acetate | 0.30 | 6.0 | 0.45 | 43.41 |
| Example 34 | 0.84 | 9.0 | Diisopropyl Adipate | 0.30 | 6.0 | 0.45 | 43.41 |
| Example 35 | 0.84 | 9.0 | cetyl acetate | 0.30 | 6.0 | 0.45 | 43.41 |
| Example 36 | 0.84 | 9.0 | JARCOL I-16 | 0.30 | 6.0 | 0.45 | 43.41 |
| Example 37 | 0.84 | 9.0 | JARCOL I-20 | 0.30 | 6.0 | 0.45 | 43.41 |
| Example 38 | 0.84 | 9.0 | PPG-2 Myristal ether propionate | 0.30 | 6.0 | 0.45 | 43.41 |
| Example 39 | 0.84 | 9.0 | Octyl dodecyl neopentanate | 0.30 | 6.0 | 0.45 | 43.41 |
| Example 40 | 0.84 | 9.0 | Acetyl tributyl citrate | 0.30 | 6.0 | 0.45 | 43.41 |
| Example 41 | 0.84 | 9.0 | Acetyl tributyl citrate | 0.30 | 6.0 | 0.45 | 43.41 |
| Example 42 | 0.84 | 9.0 | Dibutyl Sebacate | 0.30 | 6.0 | 0.45 | 43.41 |
| Example 43 | 0.84 | 9.0 | glycereth-7 benzoate | 0.30 | 6.0 | 0.45 | 43.41 |
| Example 44 | 0.84 | 9.0 | glycereth-7 triacetate | 0.30 | 6.0 | 0.45 | 43.41 |
| Example 45 | 0.84 | 9.0 | Glycerol monoLaurate | 0.30 | 6.0 | 0.45 | 43.41 |
| Example 46 | 0.84 | 9.0 | Tributyl citrate | 0.30 | 6.0 | 0.45 | 43.41 |
| Example 47 | 0.84 | 9.0 | Triethyl citrate | 0.30 | 6.0 | 0.45 | 43.41 |
| Comparative | 0.84 | 9.0 | None | 0 | 0 | 0.45 | 49.71 |
| Example 3 | | | | | | | |
| Comparative | 0.84 | 9.0 | None | 0 | 6.0 | 0.45 | 43.71 |
| Example 4 | | | | | | | |

### Antimicrobial and Cytotoxic Effects of the Compositions of Examples 29-47

The compositions of Examples 29-47 were tested according to Test Method 2, Test Method 3, and Test Method 4 described hereinabove. The bacterial strain used in Test Method 2 was Methicillin-Resistant Staphylococcus aureus (C3). The biofilms were incubated for 72 hours before contact with the test compositions. The results of the tests are shown in Table 5.

TABLE 5
Antimicrobial and Cytotoxic Effects of
the Compositions of Examples 29-47.

| | | Wounded Skin | PVM |
|---|---|---|---|
| Example | LRV¹ | Cytotoxicity² | Cytotoxicity³ |
| Example 29 | 1.1 | 25.6 | 14.4 |
| Example 30 | 1.3 | 57.6 | 69.6 |
| Example 32 | 1.4 | 46.3 | 79.0 |
| Example 32 | 2.3 | 53.2 | 85.5 |
| Example 33 | 0.8 | 56.3 | 110.5 |
| Example 34 | 0.9 | 40.2 | 51.9 |
| Example 35 | 1.3 | 48.9 | 94.7 |
| Example 36 | 0.9 | 37.3 | 97.6 |
| Example 37 | 1.0 | 49.9 | 90.1 |
| Example 38 | 1.5 | 48.6 | 75.2 |
| Example 39 | 0.9 | 40.1 | 78.2 |
| Example 40 | 1.8 | 57.2 | 72.2 |
| Example 41 | 1.1 | 47.5 | 55.4 |
| Example 42 | 0.9 | 47.8 | 66.4 |
| Example 43 | 1.0 | 38.9 | 88.4 |
| Example 44 | 0.8 | 61.6 | 86.1 |
| Example 45 | 0.9 | 49.8 | 98.0 |
| Example 46 | 1.1 | 45.2 | 92.4 |
| Example 47 | 1.1 | 37.5 | 90.2 |
| Comparative Example 3 | 0.0 | 80.2 | 107.5 |
| Comparative Example 4 | 0.9 | 47.2 | 73.9 |

¹LRV = Log Reduction Value. Determined according to Test Method 2. Colony counts from biofilms treated with antimicrobial compositions were compared to colony counts from untreated biofilms (control). The difference between control and the test compositions was reported as the LRV.
²Cytotoxicity results are reported as percent viability of the cells in the explants of Test Method 4.
³Cytotoxicity results are reported as percent viability of the cells in the explants of Test Method 3.

The results indicate the composition with an emulsifier, but no antimicrobial component or cidatrope (Comparative Example 3) had no antimicrobial activity and little or no cytotoxic effects. In addition, the composition with an emulsifier and the antimicrobial component but no cidatrope (Comparative Example 4) had nominal antimicrobial effect and was moderately cytotoxic. The data also show a number of cidatrope-containing compositions that exhibited increased antimicrobial activity and markedly less cytotoxic affects than other formulations.

### Examples 48-59. Antimicrobial Compositions Comprising Various Emulsifiers

The compositions of Examples 48-59 were prepared as described above for Examples 1-28. The compositions of these Examples are listed in Table 6.

TABLE 6
Antimicrobial compositions with various emulsifiers. All compositions in Table 6 were prepared
using PHMB as the antimicrobial component, Hydroxypropyl Guar as the thickener, and polyglycerol-3
as the polyhydric alcohol. A 60 g quantity of each composition was prepared.

| | | | Cidatrope | 20% | | | |
|---|---|---|---|---|---|---|---|
| | Hydroxypropyl | | Octyldodecanol | PHMB | | Emulsifier | |
| Example | Guar | Polyglycerol-3 | (g) | in H₂O | Emulsifier | (g) | Water |
| Example 48 | 0.90 | 9.0 | 0.30 | 1.20 | TL-10 | 1.50 | 47.10 |
| Example 49 | 0.90 | 9.0 | 0.30 | 1.20 | Decaglyn 1L | 1.50 | 47.10 |
| Example 50 | 0.90 | 9.0 | 0.30 | 1.20 | Hexaglyn - 1L | 1.50 | 47.10 |
| Example 51 | 0.90 | 9.0 | 0.30 | 1.20 | BPS-10 | 1.50 | 47.10 |
| Example 52 | 0.90 | 9.0 | 0.30 | 1.20 | LMT | 1.50 | 47.10 |
| Example 53 | 0.90 | 9.0 | 0.30 | 1.20 | Barcleanse LEP | 1.50 | 47.10 |
| Example 54 | 0.90 | 9.0 | 0.30 | 1.20 | Decaglyn 1L | 0.45 | 48.15 |
| Example 55 | 0.90 | 9.0 | 0.30 | 1.20 | PBC-34 | 1.50 | 47.10 |
| Example 56 | 0.90 | 9.0 | 0.30 | 1.20 | DOP-8N | 1.50 | 47.10 |
| Example 57 | 0.90 | 9.0 | 0.30 | 1.20 | ECT-3NEX | 1.50 | 47.10 |
| Example 58 | 0.90 | 9.0 | 0.30 | 1.20 | LSA-F | 1.50 | 47.10 |
| Example 59 | 0.90 | 9.0 | 0.30 | 1.20 | AM-3130N | 1.50 | 47.10 |

### Antimicrobial and Cytotoxic Effects of the Compositions of Examples 48-59.

The compositions of Examples 48-59 were tested according to Test Method 2 and Test Method 3 described hereinabove. The bacterial strain used in Test Method 2 was Methicillin-Resistant Staphylococcus aureus (C3). The biofilms were incubated for 72 hours before contact with the test compositions. The antimicrobial and cytotoxic activities of the compositions of Examples 48-59 were compared to a commercial antimicrobial wound gel (PRONTOSAN® Wound Gel).

TABLE 7
Antimicrobial and Cytotoxic Effects of
the Compositions of Examples 48-59.

| | | PVM² |
|---|---|---|
| Example | LRV¹ | Cytotoxicity |
| Example 48 | 3.9 | 61.3 |
| Example 49 | 4.7 | 28.0 |
| Example 50 | 6.2 | 48.9 |
| Example 51 | 6.0 | 40.7 |
| Example 52 | 1.9 | 2.8 |
| Example 53 | 2.0 | 79.1 |
| Example 54 | 5.0 | 40.4 |
| Example 55 | 6.6 | 7.0 |
| Example 56 | 2.0 | 60.8 |
| Example 57 | 2.2 | 5.1 |
| Example 58 | 2.1 | 2.8 |
| Example 59 | 4.1 | 4.5 |
| PRONTOSAN Wound Gel | 2.0 | 81.0 |

¹LRV = Log Reduction Value. Determined according to Test Method 2. Colony counts from biofilms treated with antimicrobial compositions were compared to colony counts from untreated biofilms (control). The difference between control and the test compositions was reported as the LRV.
²Cytotoxicity results are reported as percent viability of the cells in the explants of Test Method 3.

The results indicate the compositions all had antimicrobial activity. The surfactants in the compositions contributed varying degrees of cytotoxicity.

### Examples 60-66. Antimicrobial Compositions Comprising Metal Salts

The compositions of Examples 60-66 were prepared as described above for Examples 1-28. The compositions of these Examples are listed in Table 8.

The metal salts (“matrix salts”) solution was prepared by adding KCl (48.4 g; 10,00 ppm). ZnCl₂ (0.48 g; 100 ppm), RbCl (4.84 g; 1.000 ppm) and CaCl₂ (0.2 g; 42 ppm) to 3946.07 g of sterile water.

TABLE 8
Antimicrobial compositions with various emulsifiers. All compositions in Table 6 were
prepared using PHMB as the antimicrobial component, Octyldodecanol as the cidatrope,
Decaglyceryl monolaurate as the emulsifier, cross-linked PVP as the thickener, and polyglycerol-3
as the polyhydric alcohol. A 60 g quantity of each composition was prepared.

| | | | | 20% | | | Water + |
|---|---|---|---|---|---|---|---|
| | Cross-linked | | Decaglyceryl | PHMB | Monosodium | | Matrix |
| | PVP | Polyglycerol-3 | Monolaurate | in H₂O | Phosphate | Water | Salts |
| Example | (g) | (g) | (g) | (g) | (g) | (g) | (g) |
| Example 60 | 1.80 | 9.0 | 0.45 | 1.20 | 0.00 | 47.25 | — |
| Example 61 | 1.80 | 9.0 | 0.45 | 1.20 | 0.00 | — | 47.25 |
| Example 62 | 1.80 | 9.0 | 0.45 | 0.30 | 0.06 | 48.09 | — |
| Example 63 | 1.80 | 9.0 | 0.45 | 0.30 | 0.06 | — | 48.09 |
| Example 64 | 1.80 | 9.0 | 0.45 | 0.30 | 0.00 | 48.15 | — |
| Example 65 | 1.80 | 9.0 | 0.45 | 1.20 | 0.06 | 47.19 | — |
| Example 66 | 1.80 | 9.0 | 0.45 | 1.20 | 0.06 | — | 47.19 |

### Antimicrobial and Cytotoxic Effects of the Compositions of Examples 60-66

The compositions of Examples 60-66 were tested according to Test Method 1, Test Method 3, and Test Method 4 described hereinabove. The bacterial strains used in Test Method 1 were Acinetobacter baumannii (QE613) and Methicillin-Resistant Staphylococcus aureus (LAC). The results are shown in Table 9.

TABLE 9
Antimicrobial and Cytotoxic Effects of the Compositions of Examples 60-66.

| | | | PVM | 1 Day Human | 3 Day Human |
|---|---|---|---|---|---|
| Example | LRV-A¹ | LRV-B¹ | Cytotoxicity² | Skin Cytotoxicity³ | Skin Cytotoxicity³ |
| Example 60 | 4.7 | 7.7 | 45.8 | 48.6 | 24.9 |
| Example 61 | 5.1 | 7.0 | 53.9 | 24.9 | 26.2 |
| Example 62 | 1.6 | 3.7 | 69.1 | 88.9 | 55.4 |
| Example 63 | 1.9 | 3.6 | 69.1 | 79.4 | 53.1 |
| Example 64 | 2.7 | 3.7 | 77.9 | 62.4 | 47.2 |
| Example 65 | 4.8 | 8.0 | 51.1 | 53.1 | 7.4 |
| Example 66 | 5.2 | 8.4 | 42.1 | 48.4 | 7.3 |

¹LRV = Log Reduction Value. Determined according to Test Method 2. Colony counts from biofilms treated with antimicrobial compositions were compared to colony counts from untreated biofilms (control). The difference between control and the test compositions was reported as the LRV. The microorganism used for LRV-A was Acinetobacter baumannii. The bacterial survival in LRV-A was measured after 72 hours of infection followed by 24 hours exposure to the compositions. The microorganism used for LRV-B was Methicillin-Resistant Staphylococcus aureus. The bacterial survival in LRV-B was measured after 72 hour infection followed with 24 hours of exposure to the compositions.
²Cytotoxicity results are reported as percent viability of the cells in the explants of Test Method 3.
³Cytotoxicity results are reported as percent viability of the cells in the explants of Test Method 4 after 1-day exposure and 3-day exposure, respectively, to the antimicrobial compositions.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein.

1. A composition, comprising:
an antimicrobial component selected from the group consisting of octenidine, poly(hexamethylene biguanide), a quaternary ammonium antimicrobial compound, and a salt of any one of the foregoing antimicrobial components;
an emulsifying agent;
a cidatrope component;
with the proviso that the cidatrope component is not an alpha-hydroxy acid, a beta-hydroxy acid, a chelating agent, a (C1-C4)alkyl carboxylic acid, a (C6-C12)aryl carboxylic acid, a (C6-C12)aralkyl carboxylic acid, a (C6-C12)alkaryl carboxylic acid, a phenolic compound, a (C1-C10)monohydric alkyl alcohol, or an ether glycol.

2. The composition of claim 1, further comprising 1-99.5 wt % water.

3. The composition of claim 1, wherein the composition is substantially free of water and C2-C5 lower alcohols.

4. The composition of claim 1, further comprising a polyhydric glycol having an average molecular mass of about 90-3000.

5. The composition of claim 4, wherein the polyhydric glycol is selected from the group consisting of glycerol, diglycerol, polyglycerol-3, polyglycerol-4, polyglycerol-6, decaglycerol, polyglycerol-30, polyethylene glycol having a molecular weight of about 300-1000 daltons, propylene glycol, dipropylene glycol, and an ethoxylate of sorbitol, an ethoxylate of glycerol, and a combination of any two or more of the foregoing polyhydric glycols.

6. The composition of claim 1, further comprising a thickening agent.

7. The composition of claim 6, wherein the thickening agent comprises a hydrophilic polymer or an associative thickener.

8. The composition of claim 7, wherein the hydrophilic polymer is selected from the group consisting of cross-linked PVP, PVA, Guar gum, cellulose and water-soluble or water-dispersible derivatives thereof

9. The composition of claim 1, wherein the cidatrope component is selected from the group consisting of polyoxyethylene (2) lauryl ether, octyl dodecyl neopentanate, acetyl triethyl citrate, propylene glycol monocaprylate, butyl tri-n-hexyl citrate, 2-octyldecanol, 2-butyloctanol, 2-butyldecanol, 2-hexyloctanol, isocetyl alcohol, isomyristyl alcohol, isoarachidyl alcohol, hexyldecanol, isostearyl alcohol, octyldodecanol, 2-butyloctanoic acid, hexyldecanoic acid, 1,2-octanediol, ethylhexylglycerin, diisopropyladipate, propylene glycol monolaurate, capryl pyrrollidone, lauryl pyrrolidone, and triethyl citrate.

10. The composition of claim 9, wherein the cidatrope comprises a molecule having a total of at least 14 carbon atoms.

11. The composition of claim 1, wherein the quaternary ammonium antimicrobial compound or salt thereof is selected from the group consisting of Alexidine, Benzalkonium Chloride, Benzethonium Chloride, Benzyldimethylhexadecylammonium Chloride, Benzyldimethyltetradecylammonium Chloride, Cetylpyridinium Chloride, Cetyltrimethylammonium Bromide, Cetyltrimethylammonium p-Toluenesulfonate, Chloramine-T Trihydrate, Dequalinium Chloride, Dodecyltrimethylammonium Bromide, Dodecyltrimethylammonium Chloride, Domiphen Bromide, Ethylhexadecyldimethylammonium Bromide, Hexadecyltrimethylammonium Chloride, Hexamidine Diisethionate, Icthammol, Methylbenzethonium Chloride, and Tetradecyltrimethylammonium Bromide.

12. The composition of claim 1, wherein the emulsifying agent has a hydrophilic-lipophilic balance greater than 6.

13. The composition of claim 1, wherein the emulsifying agent is selected from the group consisting of decaglyceryl monolaurate, a polyoxyethylene sorbitan fatty acid ester, polyglyceryl-6 laurate, PEG-10 Phytosterol, PPG-4-Ceteth-20, hexaglycerol monolaurate, hexaglyceryl monomyristate, hexaglycerol monooleate, hexaglycerol monostearate, decaglycerol monolaurate, decaglyceryl monomyristate, decaglycryl monooleate, decaglyceryl monostearate, a PEG sorbitol ester, a PEG sorbitan ester, a PEG alkyl ester, a PEG alkyl ether, PEG-5 Soya Sterol, PEG-10 Soya Sterol, PEG-20 Soya Sterol, PEG-30 Soya Sterol, PEG-25 Phytosterol, Dihdrocholeth-30, a PEG-PPG copolymers, an alkyl ether of PEG-PPG, and an alkyl polyglucoside.

14. The composition of claim 1, further comprising a mixture of metal salts comprising KCl, ZNCl₂, RbCl, and CaCl₂.

15. The composition of claim 1, wherein the composition has a pH of about 6-7.

16. A method of treating a wound, the method comprising:
applying a composition of claim 1 to a wound site.

17. The method of claim 16, wherein applying the composition to the wound site comprises spraying the composition onto the wound site.

18. The method of claim 16, further comprising applying a wound dressing to the wound site.

19. The method of claim 16, further comprising contacting the composition to a wound dressing and contacting the wound dressing to the wound site, wherein wound site is in contact with the composition.

20. A kit comprising a composition of claim 1; and
a spray applicator, or
a wound dressing.

21-22. (canceled)
